(19) United States
(12) Patent Application Publication
(10) Pub. No.: US 2009/0144861 A1
(43) Pub. Date: Jun. 4, 2009

(54) Fungicides and Bioregulatory Mixtures

(75) Inventors: Martin Semar, Gleiszellen-Gleishorbach (DE); Dieter Strobel, Herxheim am Berg (DE); Jens Bruns, Neustadt (DE); Reinhard Stierl, Freinsheim (DE); Frank Werner, Neustadt (DE)

Correspondence Address: BIRCH STEWART KOLASCH & BIRCH, PO BOX 747, FALLS CHURCH, VA, 22040-0747, US

(73) Assignee: BASF Aktiengesellschaft, Ludwigshafen (DE)

(21) Appl. No.: 11/992,890

(22) PCT Filed: Sep. 29, 2006
(86) PCT No.: PCT/EP2006/066870

§ 371 Date: Mar. 31, 2008

(30) Foreign Application Priority Data
Oct. 7, 2005 (DE) 10 2005 048 430.1

(51) Int. Cl. A01H 5/00; A01N 43/653

(52) U.S. Cl. 800/298; 514/383; 504/139

(57) ABSTRACT

Fungicidal and bioregulatory mixtures, comprising

(1) epoxiconazole of the formula I

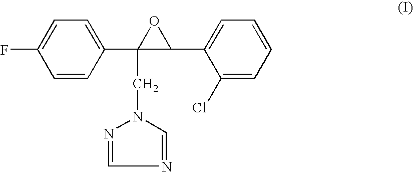

or salts or adducts thereof
and
(2) prohexadione-calcium of the formula II

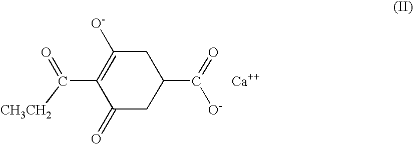

in a synergistically effective amount, methods for controlling pathogenic harmful fungi and for regulating the growth of plants using mixtures of a compound I and at least one active compound II and the use of a compound I with active compounds II for preparing such mixtures, and also compositions comprising such mixtures.

The present invention relates to fungicidal and bioregulatory mixtures comprising

(1) epoxiconazole of the formula I

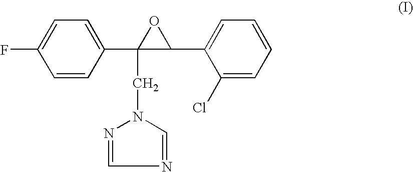

or salts or adducts thereof
and

(2) prohexadione-calcium of the formula II

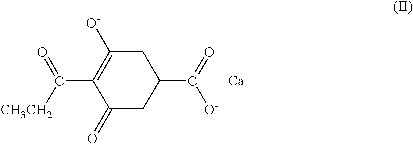

in a synergistically effective amount.

Moreover, the invention relates to a method for controlling harmful fungi using mixtures of the compound I with the compound II and to the use of the compound I and the compound II for preparing such mixtures and to compositions comprising these mixtures.

Moreover, the invention relates to a method for regulating the growth of plants using mixtures of the compound I with the compound II and to the use of the compound I and the compound II for preparing such mixtures and to compositions comprising these mixtures.

Epoxiconazole of the formula I and its use as a crop protection agent are described in EP-B 196 038.

In addition to fungicidal properties, azoles frequently also have growth-regulating properties.

Prohexadione-calcium of the formula II and its plant growth-regulating action are described in EP-A 0 123 001.

Prohexadione-calcium also has resistance-inducing properties against plant diseases in a number of plant species.

It is an object of the present invention, with a view to reducing the application rates and broadening the activity spectrum of the known compounds, to provide mixtures which, at a reduced total amount of active compounds applied, have improved activity against harmful fungi, in particular for specific indications.

We have found that this object is achieved by the mixtures defined at the outset. Moreover, we have found that simultaneous, that is joint or separate, application of the compound I and an active compound II or successive application of the compound I and an active compound II allows for better control of harmful fungi than is possible with the individual compounds (synergistic mixtures). The compound I can be used as synergist for a large number of different active compounds. By simultaneous joint or separate application of the compound I with an active compound II, the fungicidal activity is increased in a superadditive manner.

Epoxiconazole of the formula I

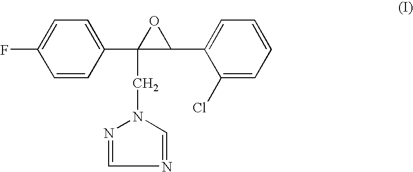

is known from EP-B 0 196 038.

Prohexadione-calcium of the formula II

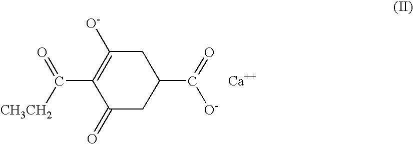

is described in EP-A 0 123 001.

Owing to the basic character of its nitrogen atoms, the compound I is capable of forming salts or adducts with inorganic or organic acids or with metal ions.

Examples of inorganic acids are hydrohalic acids, such as hydrogen fluoride, hydrogen chloride, hydrogen bromide and hydrogen iodide, sulfuric acid, phosphoric acid and nitric acid.

Suitable organic acids are, for example, formic acid, carbonic acid, and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulfonic acids (sulfonic acids having straight-chain or branched alkyl radicals of 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphoric acid radicals), where the alkyl or aryl radicals may carry further substituents, for example p-toluenesulfonic acid, salicylic acid, p-aminosalicylic acid, 2-phenoxybenzoic acid, 2-acetoxybenzoic acid, etc.

Suitable metal ions are in particular the ions of the elements of the second main group, in particular calcium and magnesium, of the third and fourth main group, in particular aluminum, tin and lead and also of transition groups one to eight, in particular chromium, manganese, iron, cobalt, nickel, copper, zinc, and others. Particular preference is given to the metal ions of the elements of transition groups of the fourth period. The metals can be present in the various valencies that they can assume.

The mixtures of the compound I and an active compound II, or the simultaneous, that is joint or separate, use of the compound I and an active compound II, have excellent activity against a broad spectrum of phytopathogenic fungi, in particular from the classes of the Ascomycetes, Deuteromycetes, Peronosporomycetes (syn. Oomycetes) and Basidiomycetes. Some of them are systemically active and can be used in crop protection as fungicides for seed dressing, as foliar fungicides and as soil fungicides.

They are particularly important for controlling a multitude of fungi on various cultivated plants, such as bananas, cotton, vegetable species (for example cucumbers, beans, tomatoes and cucurbits), barley, grass, oats, coffee, potatoes, corn, fruit species, rice, rye, soybeans, grapevines, wheat, ornamental plants, sugar cane and also on a large number of seeds.

They are especially suitable for controlling the following plant diseases:
Alternaria species on vegetable species, oilseed rape, sugar beet and fruit and rice, such as, for example, A. solani or A. alternata on potatoes and tomatoes,
Aphanomyces species on sugar beet and vegetable species,
Ascochyta species on cereals and vegetable species,
Bipolaris and Drechslera species on corn, cereals, rice and lawn, such as, for example, D. maydis on corn,
Blumeria graminis (powdery mildew) on cereals,
Botrytis cinerea (gray mold) on strawberries, vegetable species, flowers and grapevines,
Bremia lactucae on lettuce,
Cercospora species on corn, soybeans, rice and sugar beet,
Cochliobolus species on corn, cereals, rice, such as, for example, Cochliobolus sativus on cereals, Cochliobolus miyabeanus on rice,
Colletotricum species on soybeans and cotton,
Drechslera species, Pyrenophora species on corn, cereals, rice and lawn, such as, for example, D. teres on barley or D. tritici-repentis on wheat,
Esca on grapevines, caused by Phaeoacremonium chlamydosporium, Ph. Aleophilum, and Formitipora punctata (syn. Phellinus punctatus),
Elsinoe ampelina on grapevines,
Exserohilum species on corn,
Erysiphe cichoracearum and Sphaerotheca fuliginea on cucumber species,
Fusarium and Verticillium species on various plants, such as, for example, F. graminearum or F. culmorum on cereals or F. oxysporum on a large number of plants, such as, for example, tomatoes,
Gaeumanomyces graminis on cereals,
Gibberella species on cereals and rice (for example Gibberella fujikuroi on rice),
Glomerella cingulata on grapevines and other plants,
Grainstaining complex on rice,
Guignardia budwelli on grapevines,
Helminthosporium species on corn and rice,
Isariopsis clavispora on grapevines,
Michrodochium nivale on cereals,
Mycosphaerella species on cereals, bananas and peanuts, such as, for example, M. graminicola on wheat or M. fijiensis on bananas,
Peronospora species on cabbage and bulbous plants, such as, for example, P. brassicae on cabbage or P. destructor on onion,
Phakopsara pachyrhizi and Phakopsara meibomiae on soybeans,
Phomopsis species on soybeans and sunflowers, P. viticola on grapevines,
Phytophthora infestans on potatoes and tomatoes,
Phytophthora species on various plants, such as, for example, P. capsici on bell-peppers,
Plasmopara viticola on grapevines,
Podosphaera leucotricha on apple,
Pseudocercosporella herpotrichoides on cereals,
Pseudoperonospora on various plants, such as, for example, P. cubensis on cucumber or P. humili on hops,
Pseudopezicula tracheiphilai on grapevines,
Puccinia species on various plants, such as, for example, P. triticina, P. striformins, P. hordei or P. graminis on cereals, or P. asparagi on asparagus,
Pyricularia oryzae, Corticium sasakii, Sarocladium oryzae, S. attenuatum, Entyloma oryzae on rice,
Pyricularia grisea on lawn and cereals,
Pythium spp. on lawn, rice, corn, cotton, oilseed rape, sunflowers, sugar beet, vegetable species and other plants, such as, for example, P. ultiumum on various plants, P. aphanidermatum on lawn,
Rhizoctonia species on cotton, rice, potatoes, lawn, corn, oilseed rape, potatoes, sugar beet, vegetable species and on various plants, such as, for example, R. solani on beets and various plants,
Rhynchosporium secalis on barley, rye and triticale,
Sclerotinia species on oilseed rape and sunflowers,
Septoria tritici and Stagonospora nodorum on wheat,
Erysiphe (syn. Uncinula) necator on grapevines,
Setospaeria species on corn and lawn,
Sphacelotheca reilinia on corn,
Thievaliopsis species on soybeans and cotton,
Tilletia species on cereals,
Ustilago species on cereals, corn and sugar cane, such as, for example, U. maydis on corn,
Venturia species (scab) on apples and pears, such as, for example, V. inaequalis on apple.

The mixtures of the compound I and an active compound II are particularly suitable for controlling harmful fungi from the class of the Peronosporomycetes (syn. Oomycetes), such as Peronospora species, Phytophthora species, Plasmopara viticola and Pseudo-peronospora species, in particular the corresponding species mentioned above.

The mixtures of the compounds I and II are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compound I is applied by treating the fungi or the plants, seed, materials or the soil to be protected against fungal attack with a fungicidally effective amount of the active compounds. Application can be both before and after the infection of the materials, plants or seeds by the fungi.

The compound I and active compound II can be applied simultaneously, that is jointly or separately, or in succession, the sequence, in the case of separate application, generally not having any effect on the result of the control measures.

The mixtures of the compound I with the active compound II, or the simultaneous, that is joint or separate, use of a compound I and the active compound II, also have an excellent bioregulatory effect on various cultivated plants, such as bananas, cotton, vegetable species (for example cucumbers, beans, tomatoes and cucurbits), barley, grass, oats, coffee, potatoes, corn, fruit species, rice, rye, soybeans, grapevines, wheat, ornamental plants, sugar cane and also on a large number of seeds.

The present invention also provides the use of the mixtures according to the invention as a bioregulator in a number of different possible applications, for example in the cultivation of plants, e.g. in agriculture and in horticulture.

Bioregulatory active compounds are capable, for example, of influencing plant growth (growth regulators).

An example of a bioregulatory application is influencing the elongation of the aerial part of the plant (growth-regulatory). This extends to virtually all of the developmental stages of a plant.

Thus, for example, it is possible to severely inhibit the vegetative growth of the shoot of plants, which manifests itself in particular in reduced elongation. Accordingly, the growth of the treated plants is stunted; also, the leaves are darker in color. Advantageous for practice conditions is a reduced intensity of the growth of grasses on verges, hedges, canal embankments and on lawned areas such as parks, sports grounds and orchards, ornamental lawns and airports, so that grass cutting, which is laborious and expensive, can be reduced. Also, more compact growth is desirable in a number of ornamental species.

Increasing the standing ability of crops which are prone to lodging, such as cereals, corn, oilseed rape and sunflowers, is also of economic interest. The resultant shortened and strengthened stem axis reduces or eliminates the danger of “lodging” (breaking) of plants under adverse weather conditions before harvesting. Another important aspect is the growth-regulatory application for inhibiting elongation and for modifying the course of maturation over time in cotton. This makes possible completely automated harvesting of this crop plant. In fruit trees and other trees, pruning costs can be saved by means of growth regulation. At the same time, the ratio which is achieved between vegetative growth and fruit development is more advantageous. Moreover, biennial bearing of fruit trees may be avoided by means of growth regulation. Also, the growth-regulatory application may increase or inhibit lateral branching of the plants. This is of interest if, for example in tobacco plants, the development of side shoots (lateral shoots) is to be inhibited in order to favor foliar growth.

Also, frost hardness may be increased substantially by means of growth regulation, for example in the case of winter oilseed rape. Here, the vegetative development of the young oilseed rape plants after sowing and before the onset of winter frost is slowed down despite favorable growth conditions. Elongation and the development of too lush a foliar or plant biomass (which is therefore particularly sensitive to frost) are inhibited. Thus, the risk of frost damage of plants which tend toward premature breakdown of floral inhibition and tend to switch over to the generative phase is also reduced. In other crops too, for example in winter cereals, it is advantageous for the stands to be well into the tillering phase in autumn owing to growth-regulatory treatment, but to enter the cold season without unduly lush growth. This prevents increased sensitivity to subzero temperatures and—owing to the relatively low quantity of foliar biomass or plant biomass—attack by a variety of diseases (for example fungal disease). Moreover, inhibiting the vegetative growth makes possible denser planting of the soil in a number of crop plants so that higher yields based on the acreage can be achieved.

Moreover, higher yields both in terms of plant parts and in terms of plant constituents can be achieved by means of growth regulation. Thus, it is possible for example to induce the growth of larger amounts of buds, flowers, leaves, fruits, seed kernels, roots and tubers, to increase the sugar content in sugar beet, sugar cane and citrus fruits, to increase the protein content in cereals or soybeans or to stimulate increased latex flux in rubber trees. In this context, the active compounds may bring about increased yields by intervening in the plant metabolism or by promoting or inhibiting the vegetative and/or the generative growth. Finally, plant growth regulation may also bring about shortened or extended developmental stages or else an acceleration or delay in maturity of the harvested plant parts pre- or post-harvest.

Of economical interest is, for example, the facilitation of harvesting which is made possible by the concentration, over time, of the dehiscence or reduced adhesion to the tree in the case of citrus fruits, olives or in other varieties and cultivars of pome fruit, stone fruit and shelled fruit. The same mechanism, that is to say the promotion of the development of abscission tissue between, on the one hand, the fruit or leaf portion and, on the other hand, the shoot portion of the plant is also essential for a thoroughly controlled defoliation of useful plants such as, for example, cotton.

Moreover, growth regulation may bring about a reduction in the water consumption of plants. This is particulary important in the case of cropped areas which require artificial irrigation, which requires great financial input, for example in arid or semi-arid zones. Owing to the growth-regulatory application, the irrigation intensity may be reduced and farm economics improved. The effect of growth regulators may bring about better exploitation of the available water since, for example, the degree of stomatal opening is reduced, a thicker epidermis and cuticula are formed, root penetration into the soil is improved, the transpiring leaf surface area is reduced, or the microplant climate in the crop stand is advantageously affected by more compact growth.

The use according to the invention is particularly important for ornamentals, especially for fruit trees, and in particular for oilseed rape.

In a number of different possible applications in the cultivation of plants both in agriculature and in horticulture, the use of the mixture according to the invention as a bioregulator has advantages compared to the individual active compounds. In the context of the combined application according to the invention, it is possible in particular to reduce the application rates, required for bioregulation, of the individual active compounds. In addition, advantageous and especially selected added auxiliaries frequently provide better biological properties than the sum of the activities of the individual components in the tank mix method.

A particular subject matter of the present invention is the use of the mixture according to the invention as a bioregulator for improving root growth. The purpose of this use is predominantly the development of an increased number of root branches, longer roots and/or an increased root surface area. This improves the water and nutrient uptake capacity of the plants. This is advantageous in particular in the case of light, for example sandy, soils and/or when there is a lack of precipitation. In autumn, a larger storage root is formed in particular in winter oilseed rape to allow for more intense new growth in spring. In spring, the improved root system provides better anchorage of the shoot in the ground so that the plants' standing ability is markedly improved. In other plants, the storage root constitutes all or the major part of the plant organ to be harvested (for example other Brassicaceae such as radish, but also sugar beet, carrots or chicory).

Improved root growth is particularly advantageous when this is accompanied by a reduction of the vegetative growth, that is to say in particular with inhibited shoot elongation (shortening) and/or reduced foliar biomass or plant biomass. Accordingly, the present use is advantageously directed at a reduction of the quotient of shoot biomass to root biomass.

This use, which is directed at the root development, takes place in particular in cereal production, for example for wheat, barley, oats and rye, also corn and rice, and very particularly in the case of plants which develop storage roots, such as Brassicacea, for example radish, predominantly oilseed rape and in particular winter oilseed rape, and sugarbeet, carrots or chicory. Oilseed rape production must be mentioned in particular in this context; this is where an improved root growth is particularly effective. In practice, this application, which is directed at the development of roots, may gain particular importance under specific conditions, for example in the case of relatively dry soils and/or during the phase in which the plant develops the root system. With a simultaneous reduction of the shoot elongation, the improved root growth is particularly advantageous.

The compound I and the active compound II can be applied simultaneously, that is jointly or separately, or in succession, the sequence, in the case of separate application, generally not having any effect on the fungicidal and bioregulatory action.

When preparing the mixtures, it is preferred to employ the pure active compounds I and II, to which further active compounds against harmful fungi or against other pests, such as insects, arachnids or nematodes, or else herbicidal or further growth-regulating active compounds or fertilizers can be added.

Usually, mixtures of the compound I and the active compound II are employed. However, in certain cases mixtures of the compound I with two or, if appropriate, more active components may be advantageous.

The compound I and the active compound II are usually employed in a weight ratio of from 100:1 to 1:100, preferably from 20:1 to 1:20, in particular from 10:1 to 1:10.

The further active components are, if desired, added in a ratio of from 20:1 to 1:20 to the compound I.

Depending on the type of compound and the desired effect, the application rates of the mixtures according to the invention are, especially in the case of areas under agricultural cultivation, from 5 g/ha to 1750 giha, preferably from 10 to 1250 g/ha, in particular from 20 to 800 g/ha.

Correspondingly, the application rates for the compound I are generally from 1 to 1000 g/ha, preferably from 10 to 750 g/ha, in particular from 20 to 500 g/ha. Correspondingly, the application rates for the active compound II are generally from 1 to 750 g/ha, preferably from 1 to 500 g/ha, in particular from 1 to 300 g/ha.

In the treatment of seed, application rates of mixture are generally from 1 to 1000 g/100 kg of seed, preferably from 1 to 750 g/100 kg, in particular from 5 to 500 g/ 00 kg.

The method for controlling harmful fungi and also for regulating the growth of plants is carried out by the separate or joint application of the compound I and the active compound II or a mixture of the compound I and the active compound II by spraying or dusting the seeds, the plants or the soil before or after sowing of the plants or before or after the emergence of the plants.

The mixtures according to the invention, or the compound I and the active compound II can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable for use as surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compounds. The active compounds are employed in a purity of from 90% to 100%, preferably 95% to 100% (according to NMR spectrum).

The following are examples of formulations: 1. Products for dilution with water

A) Water-soluble concentrates (SL)

10 parts by weight of a compound according to the invention are dissolved in 90 parts by weight of water or of a water-soluble solvent. As an alternative, wetting agents or other auxiliaries are added. The active compound dissolves upon dilution with water. This gives a formulation having an active compound content of 10% by weight.

B) Dispersible concentrates (DC)

20 parts by weight of a compound according to the invention are dissolved in 70 parts by weight of cyclohexanone with addition of 10 parts by weight of a dispersant, for example polyvinylpyrrolidone. Dilution with water gives a dispersion. The active compound content is 20% by weight.

C) Emulsifiable concentrates (EC)

15 parts by weight of a compound according to the invention are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

D) Emulsions (EW, EO)

25 parts by weight of a compound according to the invention are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (e.g. Ultraturrax) and made into a homogeneous emulsion. Dilution with water gives an emulsion. The formulation has an active compound content of 25% by weight.

E) Suspensions (SC, OD)

In an agitated ball mill, 20 parts by weight of a compound according to the invention are comminuted with addition of 10 parts by weight of dispersants and wetting agents and 70 parts by weight of water or an organic solvent to give a fine active compound suspension. Dilution with water gives a stable suspension of the active compound. The active compound content in the formulation is 20% by weight.

F) Water-dispersible granules and water-soluble granules (WG, SG)

50 parts by weight of a compound according to the invention are ground finely with addition of 50 parts by weight of dispersants and wetting agents and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

G) Water-dispersible powders and water-soluble powders (WP, SP)

75 parts by weight of a compound according to the invention are ground in a rotor-stator mill with addition of 25 parts by weight of dispersants, wetting agents and silica gel. Dilution with water gives a stable dispersion or solution of the active compound. The active compound content of the formulation is 75% by weight.

2. Products to be applied undiluted

H) Dustable powders (DP)

5 parts by weight of a compound according to the invention are ground finely and mixed intimately with 95 parts by weight of finely divided kaolin. This gives a dustable product having an active compound content of 5% by weight.

J) Granules (GR, FG, GG, MG)

0.5 part by weight of a compound according to the invention is ground finely and associated with 99.5 parts by weight of carriers. Current methods are extrusion, spray-drying or the fluidized bed. This gives granules to be applied undiluted having an active compound content of 0.5% by weight.

K) ULV solutions (UL)

10 parts by weight of a compound according to the invention are dissolved in 90 parts by weight of an organic solvent, for example xylene. This gives a product to be applied undiluted having an active compound content of 10% by weight.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetting agent, tackifier, dispersant or emulsifier. However, it is also possible to prepare concentrates composed of active substance, wetting agent, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), it being possible thereby to apply formulations comprising over 95% by weight of active compound, or even to apply the active compound without additives.

Oils of various types, wetting agents, adjuvants may be added to the active compounds, even, if appropriate, not until immediately prior to use (tank mix). These agents are typically admixed with the compositions according to the invention in a weight ratio of from 1:100 to 100:1, preferably from 1:10 to 10:1.

The compounds I and II, or the mixtures or the corresponding formulations, are applied by treating the harmful fungi, the plants, seeds, soils, areas, materials or spaces to be kept free from them with a fungicidally effective amount of the mixture or, in the case of separate application, with the compounds I and II. Application can be carried out before or after infection by the harmful fungi.

The fungicidal effect of the individual compounds and the mixtures according to the invention was demonstrated by the following tests:

The active compounds were prepared separately or jointly as a stock solution with 25 mg of active compound which was made up to 10 ml using a mixture of acetone and/or DMSO and the emulsifier Uniperol® EL (wetting agent having emulsifying and dispersing action based on ethoxylated alkylphenols) in a volume ratio of solvent/emulsifier of 99 to 1. The mixture was then made up to 100 ml with water. This stock solution was diluted with the solvent/emulsifier/water mixture described to the concentration of active compounds stated below.

The visually determined percentages of infected leaf areas or the measured elongation were converted into efficacies in % of the untreated control:

The efficacy (E) is calculated as follows using Abbot's formula:

E=(1−α/β)×100

α corresponds to the fungicidal infection of the treated plants in % and

β corresponds to the fungicidal infection of the untreated (control) plants in %

An efficacy of 0 means that the degree of infection of the treated plants or the measured elongation corresponds to that of the untreated control plants; an efficacy of 100 means that the treated plants were not infected or that the treated plants showed no elongation.

The expected efficacies of mixtures of active compounds were determined using Colby's formula (Colby, S. R. “Calculating synergistic and antagonistic responses of herbicide combinations”, Weeds, 15, pp. 20-22, 1967) and compared with the observed efficacies.

E=x+y−x×y/100

E expected efficacy, expressed in % of the untreated control, when using the mixture of the active compounds A and B at the concentrations a and b

x efficacy, expressed in % of the untreated control, when using the active compound A at the concentration a

y efficacy, expressed in % of the untreated control, when using the active compound B at the concentration b

Use Example 1—Curative activity against brown rust of wheat caused by Puccinia recondita (Puccrt K1)

Leaves of potted wheat seedlings of the cultivar “Kanzler” were inoculated with a spore suspension of brown rust (Puccinia recondita). The pots were then placed in a chamber with high atmospheric humidity (90 to 95%) at 20 to 22° C. for 24 hours. During this time, the spores germinated and the germ tubes penetrated into the leaf tissue. The next day, the infected plants were sprayed to run off point with the active compound solution described above at the active compound concentration stated below. After the spray coating had dried on, the test plants were cultivated in a greenhouse at temperatures between 20 and 22° C. and 65 to 70% relative atmospheric humidity for 7 days. The extent of the rust fungus development on the leaves was then determined. The visually determined values for the percentage of infected leaf area were initially converted into a mean value and then converted into efficacies in % of the untreated control. An efficacy of 0 means the same degree of infection as in the untreated control, an efficacy of 100 means 0% infection. The expected efficacies for active compound combinations were determined using Colby's formula (Colby, S. R. “Calculating synergistic and antagonistic responses of herbicide Combinations”, Weeds, 15, pp. 20-22, 1967) and compared to the observed efficacies.

The active compound epoxiconazole was used as a commercial formulation.

| Active | | | | Activity | | |
|---|---|---|---|---|---|---|
| compound/active | | | Observed | calculated | | Extent of |
| compound | Conc. | | activity | to Colby | | synergism |
| combination | (mg/ml) | Ratio | (%) | (%) | Synergism | (%) |
| Epoxiconazole | 0.25 | | 11 | | | |
| Prohexadione-Ca | 1 | | 0 | | | |
| | 0.06 | | 0 | | | |
| Epoxiconazole + | 0.25 | 4:1 | 56 | 11 | Yes | 45 |
| Prohexadione-Ca | 0.06 | | | | | |
| Epoxiconazole + | 0.25 | 1:4 | 78 | 11 | Yes | 67 |
| Prohexadione-Ca | 1 | | | | | |

Use Example 2—Activity against mildew of wheat caused by Erysiphe [syn. Blumeria] graminis forma specialis. tritici (Erysgt P1)

Leaves of potted wheat seedlings were sprayed to run off point with an aqueous suspension having the active compound concentrations stated below. The suspension or emulsion had been prepared as described above. 24 hours after the spray coating had dried on, the plants were dusted with spores of mildew of wheat (Erysiphe [syn. Blumeria] graminis forma specialis. tritici). The test plants were then placed in a greenhouse at temperatures between 20 and 24° C. and at 60 to 90% relative atmospheric humidity. After 7 days, the extent of the mildew development was determined visually in % infection of the entire leaf area.

The visually determined values for the percentage of infected leaf area were initially converted into a mean value and then converted into efficacies in % of the untreated control. An efficacy of 0 means the same degree of infection as in the untreated control, an efficacy of 100 means 0% infection. The expected efficacies for active compound combinations were determined using Colby's formula (Colby, S. R. “Calculating synergistic and antagonistic responses of herbicide Combinations”, Weeds, 15, pp. 20-22, 1967) and compared to the observed efficacies.

| Active | | | | Activity | | |
|---|---|---|---|---|---|---|
| compound/active | | | Observed | calculated | | Extent of |
| compound | Conc. | | activity | to Colby | | synergism |
| combination | (mg/ml) | Ratio | (%) | (%) | Synergism | (%) |
| Epoxiconazole | 1 | | 33 | | | |
| | 0.25 | | 0 | | | |
| Prohexadione-Ca | 1 | | 0 | | | |
| | 0.25 | | 0 | | | |
| Epoxiconazole + | 1 | 4:1 | 89 | 33 | Yes | 56 |
| Prohexadione-Ca | 0.25 | | | | | |
| Epoxiconazole + | 0.25 | 1:4 | 44 | 0 | Yes | 44 |
| Prohexadione-Ca | 1 | | | | | |

1. A fungicidal and bioregulatory mixture, comprising
(I) epoxiconazole of the formula I 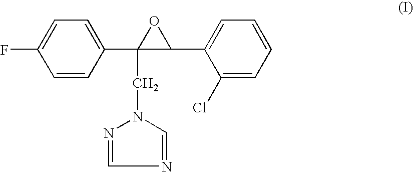
or salts or adducts thereof
and
(2) prohexadione-calcium of the formula II 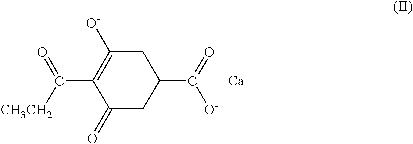
in a synergistically effective amount.

2. The fungicidal and bioregulatory mixture according to claim 1, wherein the weight ratio of epoxiconazole of the formula I to prohexadione-calcium of the formula II is from 100:1 to 1:100.

3. A method for controlling phytopathogenic harmful fungi, which comprises treating the harmful fungi, their habitat or the plants, the soil or seed to be protected against them with the fungicidal mixture according to claim 1.

4. A method for regulating the growth of plants, which comprises treating the plants, the soil or seed with the bioregulatory mixture according to claim 1.

5. The method according to claim 3, wherein epoxiconazole of the formula I and prohexadione-calcium of the formula II are applied simultaneously, that is jointly or separately, or in succession.

6. The method according to claim 3, wherein the fungicidal and bioregulatory mixture or epoxiconazole of the formula I with prohexadione-calcium of the formula II are applied in an amount of from 5 g/ha to 1750 g/ha.

7. The method according to claim 3, wherein the compounds I and II or the mixture are applied in an amount from 1 g to 1000 g per 100 kg of seed.

8. Seed, comprising the mixture according to claim 1 in an amount of from 1 g to 1000 g per 100 kg.

9. The use of the compounds I and II according to claim 1 for preparing a composition suitable for controlling harmful fungi.

10. The use of the compounds I and II according to claim 1 for preparing a composition suitable for regulating the growth of plants.

11. A fungicidal composition, comprising the fungicidal mixture according to claim 1 and a solid or liquid carrier.

12. A bioregulatory composition, comprising the mixture according to claim 1 and a solid or liquid carrier.

13. The method according to claim 4, wherein epoxiconazole of the formula I and prohexadione-calcium of the formula II are applied simultaneously, that is jointly or separately, or in succession.

14. The method wherein epoxiconazole of the formula I according to claim 1-and prohexadione-calcium of the formula II are applied simultaneously, that is jointly or separately, or in succession.

15. The method wherein epoxiconazole of the formula I and prohexadione-calcium of the formula II according to claim 1 are applied simultaneously, that is jointly or separately, or in succession.

16. The method according to claim 4, wherein the fungicidal and bioregulatory mixture or epoxiconazole of the formula I with prohexadione-calcium of the formula II are applied in an amount of from 5 g/ha to 1750 g/ha.

17. The method according to claim 5, wherein the fungicidal and bioregulatory mixture or epoxiconazole of the formula I with prohexadione-calcium of the formula II are applied in an amount of from 5 g/ha to 1750 g/ha.

18. The method wherein the fungicidal and bioregulatory mixture or epoxiconazole of the formula I with prohexadione-calcium of the formula II according to claim 1 are applied in an amount of from 5 g/ha to 1750 g/ha.

19. The method according to claim 4, wherein the compounds I and II or the mixture are applied in an amount from 1 g to 1000 g per 100 kg of seed.

20. The method according to claim 5, wherein the compounds I and II or the mixture are applied in an amount from 1 g to 1000 g per 100 kg of seed.
